(19) United States
(12) Patent Application Publication
(10) Pub. No.: US 2012/0253422 A1
(43) Pub. Date: Oct. 4, 2012

(54) SYSTEMS AND METHODS FOR SELECTING NEURAL MODULATION CONTACTS FROM AMONG MULTIPLE CONTACTS

(75) Inventors: James R. Thacker, Homer, AK (US); Jon Parker, San Jose, CA (US)

(73) Assignee: Nevro Corporation, Menlo Park, CA (US)

(21) Appl. No.: 13/076,197

(22) Filed: Mar. 30, 2011

(51) Int. Cl. A61N 1/36

(52) U.S. Cl. 607/46

(57) ABSTRACT

The present technology is directed generally to systems and methods for selecting neural modulation contacts from among multiple contacts. A system in accordance with a particular embodiment includes a patient implantable signal delivery system having (n) contacts positioned to deliver therapy signals to a patient, where (n) is greater than three, and an external signal generator coupled to the signal delivery device and having a computer-readable medium containing instructions that, when executed, perform the operations of (a) identifying a contact pair, (b) delivering neural modulation signals to the contact pair, (c) changing one or more of the contacts of the contact pair, and (d) repeating operations (b)-(c) for each of at most (n−1) unique contact pairs.

TECHNICAL FIELD

The present technology is directed generally to systems and methods for selecting neural modulation contacts from among multiple contacts, for example, during a trial period. After the trial period, the selected contacts can provide longer term neural modulation.

### BACKGROUND

Neurological stimulators have been developed to treat pain, movement disorders, functional disorders, spasticity, cancer, cardiac disorders, and various other medical conditions. Implantable neurological stimulation systems generally have an implantable pulse generator and one or more leads that deliver electrical pulses to neurological tissue or muscle tissue. For example, several neurological stimulation systems for spinal cord stimulation (SCS) have cylindrical leads that include a lead body with a circular cross-sectional shape and multiple conductive rings spaced apart from each other at the distal end of the lead body. The conductive rings operate as individual electrodes or contacts and the SCS leads are typically implanted either surgically or percutaneously through a large needle inserted into the epidural space, with or without the assistance of a stylet.

Once implanted, the pulse generator applies electrical pulses to the electrodes, which in turn modify the function of the patient's nervous system, such as by altering the patient's responsiveness to sensory stimuli and/or altering the patient's motor-circuit output. During pain treatment, the pulse generator applies electrical pulses to the electrodes, which in turn can generate sensations that mask or otherwise alter the patient's sensation of pain. For example, in many cases, patients report a tingling or paresthesia that is perceived as more pleasant and/or less uncomfortable than the underlying pain sensation. In other cases, the patients can report pain relief without paresthesia or other sensations.

In any of the foregoing systems, it is important for the practitioner to accurately position the stimulator in order to provide effective therapy. One approach for easing the burden of accurately locating the stimulator is to provide the stimulator with multiple contacts, and allow the practitioner to selectively activate and deactivate particular contacts until a suitable set of contacts is identified. For example, typical existing SCS leads include eight contacts, and in many cases the practitioner implants two such leads in order to provide a reasonable array of options from which to select a particular contact or group of contacts for extended use. One drawback with this approach is that the number of possible combinations of contacts increases exponentially with each added contact. Accordingly, the process of selecting appropriate contacts can become a burdensome and time consuming task, even for a relatively low number of contacts. As a result, there exists a need for simplified techniques and associated systems for selecting appropriate neural modulation contacts.

### BRIEF DESCRIPTION OF THE DRAWINGS

FIG. 1A is a partially schematic illustration of an implantable spinal cord modulation system positioned at a patient's spine to deliver therapeutic signals in accordance with several embodiments of the present disclosure.

FIG. 1B is a partially schematic, cross-sectional illustration of a patient's spine, illustrating representative locations for an implanted lead in accordance with embodiments of the disclosure.

FIG. 2 is a partially schematic, enlarged illustration of a representative signal delivery device configured in accordance with an embodiment of the disclosure.

FIG. 3 is a flow diagram illustrating a representative process for selecting neural modulation contacts in accordance with several embodiments of the disclosure.

FIG. 4A is a flow diagram illustrating a process for selecting neural modulation contacts in accordance with several further embodiments of the disclosure.

FIG. 4B is a partially schematic illustration of a portion of the signal delivery device shown in FIG. 2.

### DETAILED DESCRIPTION

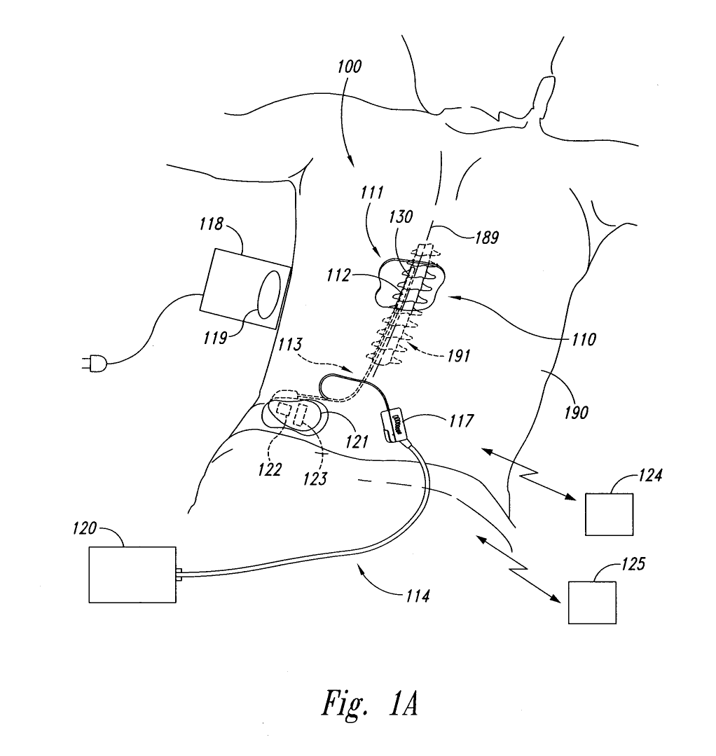

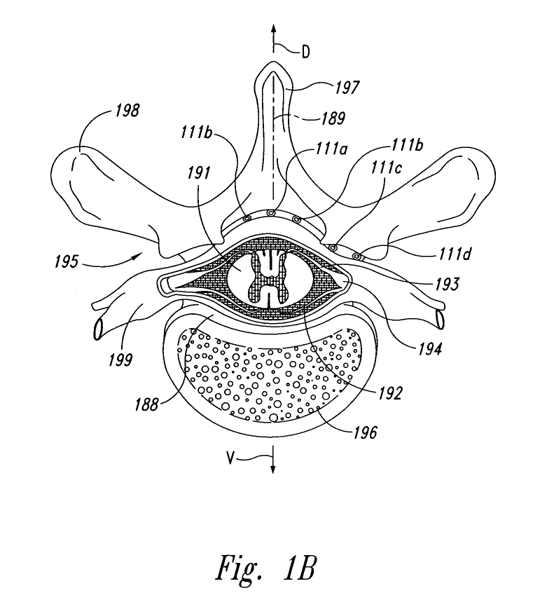

The present technology is directed generally to systems and methods for selecting implanted contacts that provide neural stimulation to a patient. In at least some contexts, the systems and methods are used during a “trial” period to select contacts proximate to the patient's spinal cord. The selected contacts are then used to deliver high frequency signals that modulate neural activity at the patient's spine, in particular embodiments, to address chronic pain over a longer period of time. In other embodiments, however, the systems and associated methods can have different configurations, components, and/or procedures. Still other embodiments may eliminate particular components and/or procedures. A person of ordinary skill in the relevant art, therefore, will understand that the present technology and its associated procedures may include other embodiments with additional elements or steps, and/or may include other embodiments without several of the features or steps shown and described below with reference to FIGS. 1A-4B.

Several aspects of the technology are embodied in computing devices; e.g., programmed pulse generators, controllers and/or other devices. The computing devices via which the described technology can be implemented may include one or more central processing units, memory, input devices (e.g., input ports), output devices (e.g., display devices), storage devices, and network devices (e.g., network interfaces). The memory and storage devices are computer-readable media that may store instructions that implement the technology. In many embodiments, the computer-readable media are tangible media. In other embodiments, the data structures and message structures may be stored or transmitted via an intangible data transmission medium, such as a signal on a communications link. Various suitable communications links may be used, including but not limited to a local area network and/or a wide-area network.

FIG. 1A schematically illustrates a representative patient system 100 for providing relief from chronic pain and/or other conditions, arranged relative to the general anatomy of a patient's spinal cord 191. The overall patient system 100 can include a signal delivery system 110, which may be implanted within a patient 190, typically at or near the patient's spinal cord midline 189, and coupled to a pulse generator 121. The signal delivery system 110 can provide therapeutic electrical signals to the patient during operation.

In a representative example, the signal delivery system 110 includes a signal delivery device 111 that carries features for delivering therapy to the patient 190 after implantation. The pulse generator 121 can be connected directly to the signal delivery device 111, or it can be coupled to the signal delivery device 111 via a signal link 113 (e.g., an extension). In a further representative embodiment, the signal delivery device 111 can include one or more elongated lead(s) or lead body or bodies 112. As used herein, the terms “lead” and “lead body” include any of a number of suitable substrates and/or support members that carry devices for providing therapy signals to the patient 190. For example, the lead or leads 112 can include one or more electrodes or electrical contacts that direct electrical signals into the patient's tissue, such as to provide for patient relief. In other embodiments, the signal delivery device 111 can include structures other than a lead body (e.g., a paddle) that also direct electrical signals and/or other types of signals to the patient 190.

The pulse generator 121 can transmit signals (e.g., electrical signals) to the signal delivery device 111 that up-regulate (e.g., stimulate or excite) and/or down-regulate (e.g., block or suppress) target nerves. As used herein, and unless otherwise noted, the terms “modulate” and “modulation” refer generally to signals that have either type of the foregoing effects on the target nerves. The pulse generator 121 can include a machine-readable (e.g., computer-readable) medium containing instructions for generating and transmitting suitable therapy signals. The pulse generator 121 and/or other elements of the system 100 can include one or more processors 122, memories 123 and/or input/output devices. Accordingly, the process of providing modulation signals, changing which contacts are active, evaluating results, selecting contacts for long-term use, and/or executing other associated functions can be performed by computer-executable instructions contained on, in or by computer-readable media located at the pulse generator 121 and/or other system components. The pulse generator 121 can include multiple portions, elements, and/or subsystems (e.g., for directing signals in accordance with multiple signal delivery parameters), carried in a single housing, as shown in FIG. 1A, or in multiple housings.

In some embodiments, the pulse generator 121 can obtain power to generate the therapy signals from an external power source 118. The external power source 118 can transmit power to the implanted pulse generator 121 using electromagnetic induction (e.g., RF signals). For example, the external power source 118 can include an external coil 119 that communicates with a corresponding internal coil (not shown) within the implantable pulse generator 121. The external power source 118 can be portable for ease of use.

In at least some cases, an external programmer 120 (e.g., a trial modulator) can be coupled to the signal delivery device 111 during an initial procedure, prior to implanting the pulse generator 121. For example, a practitioner (e.g., a physician and/or a company representative) can use the external programmer 120 to vary the modulation parameters provided to the signal delivery device 111 in real time, and select optimal or particularly efficacious parameters. These parameters can include the location from which the electrical signals are emitted (e.g., which contacts of a multi-contact signal delivery device are active and which are not), as well as the characteristics of the electrical signals provided to the signal delivery device 111. In a typical process, the practitioner uses a cable assembly 114 to temporarily connect the external programmer 120 to the signal delivery device 111. The practitioner can test the efficacy of the signal delivery device 111 in an initial position. The practitioner can then disconnect the cable assembly 114 (e.g., at a connector 117), reposition the signal delivery device 111, and reapply the electrical modulation. This process can be performed iteratively until the practitioner obtains the desired position for the signal delivery device 111. Optionally, the practitioner may move the partially implanted signal delivery element 111 without disconnecting the cable assembly 114.

After a trial period with the external programmer 120, the practitioner can implant the implantable pulse generator 121 within the patient 190 for longer term treatment. The signal delivery parameters provided by the pulse generator 121 can still be updated after the pulse generator 121 is implanted, via a wireless physician's programmer 125 (e.g., a physician's remote) and/or a wireless patient programmer 124 (e.g., a patient remote). Generally, the patient 190 has control over fewer parameters than does the practitioner.

FIG. 1B is a cross-sectional illustration of the spinal cord 191 and an adjacent vertebra 195 (based generally on information from Crossman and Neary, “Neuroanatomy,” 1995 (published by Churchill Livingstone)), along with multiple signal delivery devices 111 (shown as signal delivery devices 111a-d) implanted at representative locations. For purposes of illustration, multiple signal delivery devices 111 are shown in FIG. 1B implanted in a single patient. In actual use, any given patient will likely receive fewer than all the signal delivery devices 111 shown in FIG. 1B.

The spinal cord 191 is situated within a vertebral foramen 188, between a ventrally located ventral body 196 and a dorsally located transverse process 198 and spinous process 197. Arrows V and D identify the ventral and dorsal directions, respectively. The spinal cord 191 itself is located within the dura mater 199, which also surrounds portions of the nerves exiting the spinal cord 191, including the ventral roots 192, dorsal roots 193 and dorsal root ganglia 194. In one embodiment, a single first signal delivery device 111a is positioned within the vertebral foramen 188, at or approximately at the spinal cord midline 189. In another embodiment, two second signal delivery devices 111b are positioned just off the spinal cord midline 189 (e.g., about 1 mm. offset) in opposing lateral directions so that the two signal delivery devices 111 b are spaced apart from each other by about 2 mm. In still further embodiments, a single signal delivery device or pairs of signal delivery devices can be positioned at other locations, e.g., at the dorsal root entry zone as shown by a third signal delivery device 111c, or at the dorsal root ganglia 194, as shown by a fourth signal delivery device 111d.

In any of the foregoing embodiments, it is important that the signal delivery device 111 and in particular, the electrical contacts of the device, be placed at a target location that is expected (e.g., by a practitioner) to produce efficacious results in the patient when the device 111 is activated. The following disclosure describes techniques and systems for simplifying the process of selecting appropriate contacts via which to deliver neural modulation signals to the patient.

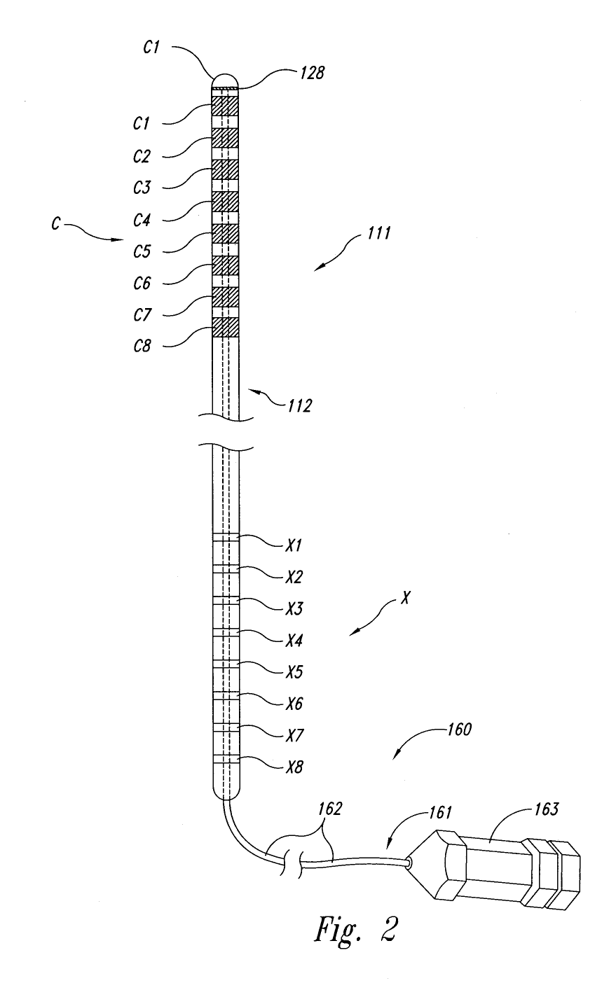

FIG. 2 is a partially schematic illustration of a representative signal delivery device 111 that includes a lead 112 having a distal region that carries a plurality of ring-shaped therapy contacts C positioned to deliver therapy signals to the patient when the lead 112 is implanted. In a representative embodiment, the lead 112 includes eight therapy contacts C, identified individually as contacts C1, C2, C3 . . . C8. The lead 112 includes internal wires or conductors (not visible in FIG. 2) that extend between the therapy contacts C at the distal region of the lead 112, and corresponding connection contacts X (shown as X1, X2, X3 . . . X8) positioned at the proximal end.

After implantation, the connection contacts X are connected to the external programmer 120 or to the implanted pulse generator 121 discussed above with reference to FIG. 1A. During implantation, an implanting tool 160 (e.g., a stylet 161) is temporarily coupled to the lead 112 to support the lead 112 as it is inserted into the patient. For example, the implanting tool 160 can include a shaft 162 that is slideably and releasably inserted (via a handle 163) into an axially-extending opening in the lead 112. The shaft 162 is generally flexible, but more rigid than the lead 112 to allow the practitioner to insert the lead 112 and control its position during implantation. A stylet stop 128 at the distal end of the lead opening prevents the practitioner from over-inserting the stylet shaft 162. In particular embodiments, the stylet stop 128 can include platinum and/or another radiopaque material that allows the practitioner to identify the location of the end of the implanted lead 112 using fluoroscopy and/or another suitable technique.

During the trial period described above, the practitioner typically varies several parameters, for example, the amplitude, frequency, pulse width, and/or polarity of the signals, and/or the contacts from which the signals are delivered. Even with only eight available contacts, and assuming the practitioner selects contacts in pairs, the number of unique contact pairs is 28. If the practitioner attempts to vary other parameters in addition to the identity of the active contacts, and/or if the practitioner delivers signals via only individual contacts or more than two contacts at a time, the number of possible combinations quickly becomes unmanageable. Aspects of the present technology are directed to reducing the practitioner's workload and/or the amount of time required to identify suitable signal delivery contacts by constraining the manner in which the contacts are selected.

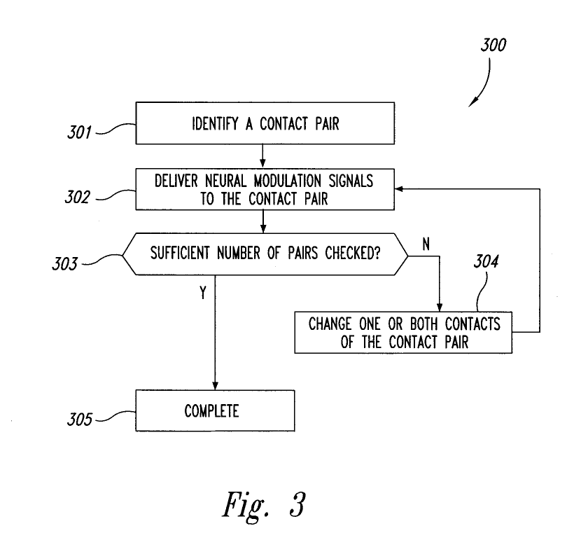

FIG. 3 is a high level flow diagram illustrating a representative process 300 in accordance with an embodiment of the present technology. The process 300 can include identifying a contact pair (process portion 301) and delivering neural modulation signals to the contact pair (process portion 302). Process portion 303 includes determining whether a sufficient number of contact pairs have been checked. If a sufficient number of contact pairs has been checked, then process portion 305 includes completing the overall selection process. For example, process portion 305 can include identifying a pair of contacts suitable for additional (e.g., extended) therapy. If a sufficient number of contact pairs has not been checked, then process portion 304 includes changing one or both contacts of the contact pair, and repeating process portion 302 until the requisite number of pairs has been checked. As will be described in further detail below, and in at least one embodiment, the suitable number of pairs can be limited or constrained to be less than the total number of available contacts.

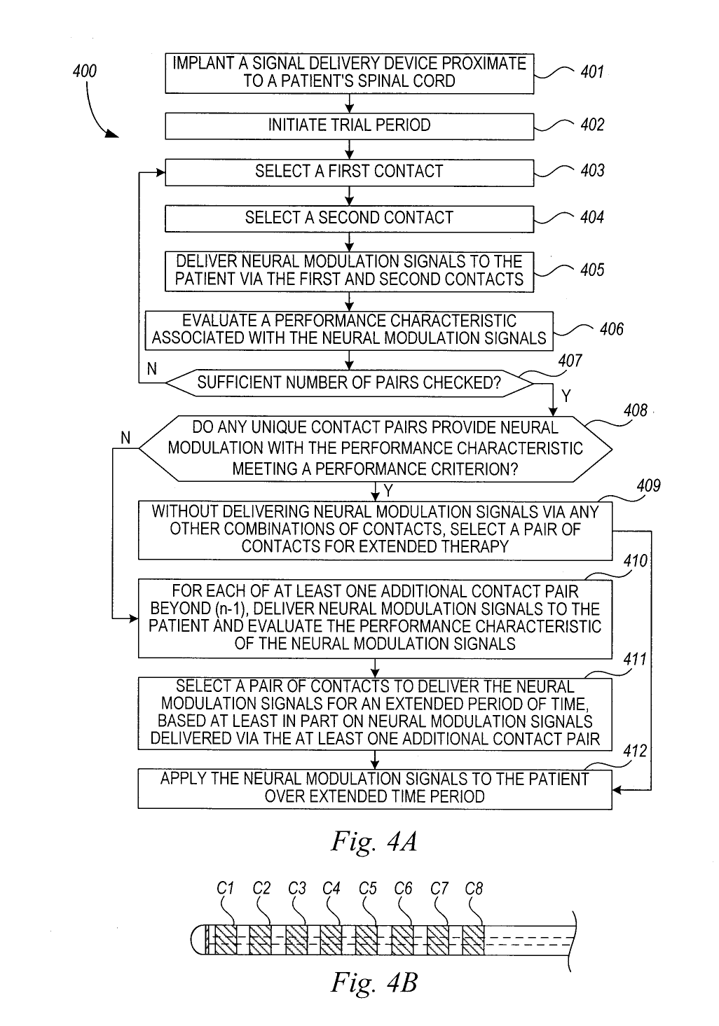

FIG. 4A is a more detailed flow diagram of a representative process 400 for identifying suitable contacts during a trial period and using the identified contacts to provide therapy to the patient over an extended period of time. For purposes of illustration, certain references are made below to the eight contacts shown in FIG. 2 and reproduced in FIG. 4B. It will be understood by those of ordinary skill in the relevant art that in other embodiments, similar processes may be used in the context of contacts other than those shown in FIGS. 2 and 4B, including greater and/or lesser numbers of contacts, and/or contacts having different configurations.

Process portion 401 includes implanting a signal delivery device proximate to the patient's spinal cord, and process portion 402 includes initiating a trial period. The signal delivery device can include one or more elongated leads with eight contacts, as shown in FIG. 4B, or in other embodiments, the signal delivery device can include other structures, including those with a higher or lower number of contacts. Process portions 403 and 404 include selecting a first contact and a second contact, respectively. Accordingly, this particular example is suitable for selecting contacts for bipolar neural modulation. In other embodiments, the practitioner may provide signals to the patient in a monopolar manner or in other manners (e.g., a tripolar manner), in which case a different number of contacts is selected during this stage of the process 400. For example, if the practitioner provides monopolar signals, then a ground or return contact (which may or may not be carried by the signal delivery device) can remain the same throughout the process. If the practitioner provides tripolar signals, then the process 400 can further include selecting a third contact.

Process portions 403 and 404 can be performed entirely manually or these process portions can be automated to varying degrees. For example, the practitioner can manually select both the first and second contacts in one embodiment. In another embodiment, the practitioner can select the first contact, and the second contact can be automatically selected using a suitable algorithm. In other embodiments, the practitioner selects neither the first nor the second contact, and both can be automatically selected using a suitable algorithm.

The identity of the second contact can depend on the identity of the first contact. For example, if the first contact is the distal-most contact C1, the second contact can include the nearest neighboring contact C2. In another embodiment, the algorithm can include skipping one or more contacts between the first contact and the second contact. Accordingly, if the distal-most contact C1 is the first contact, contact C3 can be the second contact, or contact C4 can be the second contact, etc., depending upon factors that may include the particular patient's condition and/or the total number of contacts on the lead 112 and/or the spacing between neighboring contacts. In particular embodiments, the contacts can be spaced apart by about 5 millimeters. In other embodiments, the contacts have smaller spacing intervals (e.g., about 2-3 millimeters) or larger spacing intervals (e.g., 32 millimeters, or approximately one vertebral body).

Process portion 405 includes delivering neural modulation signals to the patient via the selected first and second contacts, and process portion 406 includes evaluating a performance characteristic of the system and associated with the neural modulation signals applied in process portion 405. The performance characteristic can include the efficacy with which the neural modulation signals address the patient's condition. For example, if the patient suffers from chronic pain, the performance characteristic can include the degree to which the patient's pain is alleviated by virtue of the neural modulation signals delivered in process portion 405. In other embodiments, the performance characteristic can relate to other patient conditions, for example, patient motor performance or cognitive performance. In still further embodiments, the performance characteristic can relate to system attributes, e.g., in addition to the patient's response. If the patient's response is relatively constant, the performance characteristic can relate to system attributes instead of the patient's response. A representative system attribute is the power consumed by the system as it delivers the neural modulation signals. In any of the foregoing embodiments, the patient can track performance characteristics associated with patient sensations (e.g., pain) via a diary (electronic or otherwise), the patient's memory, or another technique. The system can automatically track system attributes and can store the results in magnetic, electronic, optical, or any other type memory.

In another representative embodiment, the system attribute can include whether or not the contact pair is defective. For example, the system can automatically determine if one of the contacts is faulty (e.g., disconnected) or otherwise deficient via an impedance technique, and can automatically substitute another contact (e.g., the nearest contact) for further testing, without intervention by the patient or practitioner.

Process portion 407 includes determining whether a sufficient number of contact pairs have been checked. In a particular embodiment, the number of contact pairs is less than the total number of available contacts. For example, a sufficient number of contact pairs can be one less than the total number of available contacts (e.g., if the total number of contacts is “n,” than a sufficient number of contact pairs can be “n−1”). If all n contacts are carried by the signal delivery device, then checking up to n−1 contacts can be suitable for monopolar or multipolar signals. If one of the n contacts is off the signal delivery device (e.g., a fixed return contact), then up to all the contacts on the signal delivery device may be checked, but (with the return contact considered one of the n contacts) checking up to n−1 contacts can still be suitable for both monopolar and multipolar signals. In several representative embodiments, n is greater than three; e.g., n is equal to eight in the embodiment shown in FIG. 4B.

If a sufficient number of contact pairs have not yet been checked, the process 400 returns to process portion 403, and process portions 403-406 are repeated until the condition of process portion 407 is met. In a representative embodiment, contacts C1 and C2 are selected as the first and second contacts initially. When the process returns to process portion 403, contacts C2 and C3 are selected. The selected pairs of contacts are incremented in this manner until contacts C7 and C8 are selected. In another embodiment, contacts C1 and C3 can be selected initially, contacts C2 and C4 on the next pass, contacts C3 and C5 on the subsequent pass, and so on until the condition of process portion 407 is met.

Process portions 403-406 can automatically be repeated in certain embodiments. For example, during at least some patient therapies, the patient does not experience paresthesia (or other potentially undesirable sensations, including pain caused by the therapy itself). Accordingly, in such a case, all the candidate contact pairs can be tested automatically without the risk of causing patient pain. In certain embodiments, the practitioner can cycle through all the candidate pairs while the patient is in the office to ensure that none of the pairs will cause pain or another undesirable sensation when selected automatically during the trial period. If this check identifies problematic contacts, the practitioner can de-select those contacts or contact combinations so that they are not tested during the trial period.

In process portion 408, the process 400 includes determining whether any of the unique contact pairs selected and evaluated in process portions 403-406 provide neural modulation with a performance characteristic meeting a selected criterion. The selected criterion may include a level of pain relief in some embodiments, and other values in other embodiments depending, e.g., upon what performance characteristic was evaluated in process portion 406. If this condition is met, then process portion 409 includes selecting the pair of contacts for extended therapy without delivering neural modulation signals via any other combinations of contacts. Accordingly, process portion 409 can include reviewing the evaluations established at process portion 406 for each of the contact pairs that are checked, and selecting the contact pair that produces the best performance characteristic. Because process portion 409 includes selecting the contact pair without delivering neural modulation signals via any further combinations of contacts, the number of tested contact pairs is limited to the sufficient number of contacts described above with reference to process portion 407. Once the selected pair of contacts has been determined in process portion 409, the process 400 continues with process portion 412 by applying the neural modulation signals to the patient over an additional (e.g., extended) period of time, via the selected contact pair. The extended period of time can include a period of weeks, months or years depending upon factors that include the patient's condition (and/or the stability of the condition) and the efficiency of the treatment. In any of these embodiments, the extended period is typically longer than the trial period.

If, in process portion 408, none of the unique contact pairs tested in repeated cycles through process portions 403-406 produces a suitable performance level, then in process portion 410, at least one additional contact pair can be selected and neural modulation signals delivered to the patient via the at least one additional contact pair. Process portion 410 further includes evaluating the performance characteristic associated with the neural modulation signals in process portion 411, the pair of contacts selected for an extended period of time is based at least in part on the evaluation of neural modulation signals delivered via the at least one additional contact pair.

Other embodiments can include variations, substitutions, additions and/or deletions relative to the foregoing processes. For example, in particular embodiments, each tested contact combination is tested for a predefined period (e.g., one day) before the next contact combination is tested. This period can be extended or reduced by the practitioner and/or the patient to account for patient responses that may differ from one patient to the next and/or one type of therapy or therapy location to the next. In another embodiment, the process can be performed in a multi-tiered manner. For example, after one pass through an initial set of contact combinations, the process can include selecting only those contact combinations that exceed a given threshold level (e.g., the performance criterion). Then only those contact combinations are tested in a second pass, and the contact combination producing a particular performance level (e.g., the best performance level) is selected for extended therapy. This process can be repeated for more than two tiers in some instances, and in any of these arrangements, each subsequent tier process can focus on selecting from only the best candidates identified in the previous tier. In still another embodiment, the contact combination producing the best performance level is automatically left “on” for extended therapy, e.g., after a single tier process, or a multi-tier process.

In yet another embodiment, the patient can initiate any of the foregoing processes of his or her own accord, e.g., during a trial period and/or after a trial period (e.g., during a post-trial, extended therapy period). For example, if the patient notices a decrease in the post-trial efficacy of the therapy, the patient can initiate a process of re-testing the contacts to identify a more efficacious contact combination. The patient can control other aspects of the process in addition to or in lieu of the foregoing initiation step. For example, the patient can automatically terminate the process once a particularly beneficial (e.g., efficacious) contact combination is identified, without continuing to test any remaining, untested contact combinations.

An advantage of several of the embodiments described above is that the number of contact pairs tested during the trial period can be constrained when compared with the total number of possible contact pair combinations. For example, it is expected that in many types of therapy, a significant number of patients may be successfully treated without engaging in process portions 410 and 411. In particular examples, it is expected that approximately 90% of the patients will receive effective treatment via the constrained trial period process described above. Because process portions 410 and 411 are conducted on only a fraction of the total number of treated patients, the overall amount of time the practitioner must spend engaging in trial therapies for a given population of patients can be significantly reduced.

Another advantage of several of the embodiments described above is that the same algorithm can be used to select contacts for patients in a variety of different conditions. For example, the same algorithm can be used for all patients suffering from chronic low back pain, without regard to the variations in pain severity from one patient to the other. In other embodiments, the same algorithm can be used for patients having different diagnoses. For example, the same algorithm can be used to select contacts in cases where the lead is implanted at the patient's thoracic vertebra (e.g., for lower back pain) as is used when the lead is implanted at the patient's cervical vertebra (e.g., for neck pain). Accordingly, the constrained number of contact combinations that are used in accordance with selected embodiments described above can allow these embodiments to be used in a wide variety of settings without the need for adjusting the algorithm from one setting to the other.

In at least some embodiments, it may not be immediately apparent to either the patient or the practitioner whether or not a particular pair (or other set) of contacts selected during the trial period is effective. For example, in at least some embodiments, the patient experiences no immediate sensation of paresthesia, or does not experience an immediate pain reduction because the pain is experienced only or primarily when the patient is active, as opposed to when the patient is in a practitioner's office. Accordingly, the patient may spend a significant period of time (e.g., one day or more) receiving therapy via a particular set of contacts before it is determined whether or not that set of contacts is effective. As a result, the practitioner can reduce this period of time by constraining the number of contact pairs or other sets that are tested during the trial period by days or even weeks.

In a particular example described above, the trial period begins with a pair of contacts selected to include the distal-most contact C1. In this embodiment, the contact pairs are changed in a sequential, monotonic manner from the distal-most contact C1 to the proximal-most contact C8. In other embodiments, the practitioner may prefer other selection algorithms, based for example on how the lead is positioned relative to the target neural population. For example, if the practitioner centers the lead at the target neural population, the trial period algorithm may be different. In a particular example, the practitioner positions the lead so that contact C5 is centered at the target neural population. In this case, the algorithm can include testing contacts C5 and C6, then contacts C5 and C4, then contacts C6 and C7, then contacts C4 and C3 so as to gradually move the signal delivery area outwardly from the initially selected location. As described above, this process can include selecting immediately neighboring contacts, or skipping selected contacts (e.g., testing contacts C5 and C7, then C5 and C3, then C6 and C8, etc.). In other embodiments, the initial contact pair (and then subsequent contact pairs) can be selected in other manners that parallel, follow, or are otherwise based on the expected probability of success for the contact pairs. In any of the foregoing embodiments, the initial contact pair can be selected automatically. For example, if the practitioner provides input to the system identifying where the lead is relative to the patient's anatomy, and the therapy is expected to produce the most effective results at the T10 vertebral level, the system can automatically select those contacts at or close to the T10 vertebral level at the outset of the process. A further aspect of this process can include eliminating particular contacts that are not expected to contribute significantly to the planned therapy. For example, the patient or practitioner can manually eliminate contacts that are distal from the target treatment site. In other embodiments, the system can automatically locate such contacts (e.g., via impedance measurements or other techniques) and can automatically eliminate such contacts. In general, the contacts that participate in the process can be selected based on data from an individual patient (e.g., the patient receiving the therapy) and/or other patients (e.g., patients with a similar diagnosis and/or treatment regiment), whether the data resides on the controller, or is obtained or accessed from a remote location.

The foregoing process may be used to identify contacts for therapy in accordance with any of a variety of therapy delivery parameters. In particular embodiments, the therapy is provided to a selected vertebral level at a relatively high frequency. For example, the signals can be provided at a frequency of from about 3 kHz to about 50 kHz, at a vertebral level of from about T9 to about T12, inclusive, to address chronic low back pain. Further details of particular signal delivery parameters associated with treating chronic patient pain via high frequency signals delivered at the foregoing vertebral levels are included in pending U.S. patent application Ser. No. 12/765,747, filed on Apr. 22, 2010 and incorporated herein by reference. It is expected that using embodiments of the selection process described above, with n−1 or fewer sets of contacts tested during a trial period, will produce a therapy that meets the performance criterion in a majority of patients over a representative patient population.

Aspects of the foregoing technology can be applied to leads and/or signal delivery devices having configurations other than those expressly described above. Representative devices are disclosed in the following pending U.S. Applications, all of which are incorporated herein by reference: Ser. Nos. 12/104,230 (filed Apr. 16, 2008); 12/468,688 (filed May 19, 2009); 12/129,078 (filed May 29, 2008); 12/562,892 (filed Sep. 18, 2009); 12/895,403 (filed Sep. 30, 2010); and 12/895,438 (filed Sep. 30, 2010). Aspects of the foregoing technology can be used in combination with other parameter selection methodologies, including those disclosed in the following pending U.S. Applications, all of which are incorporated herein by reference: Ser. Nos. 12/703,683 (filed Feb. 10, 2010); 12/499,769 (filed Jul. 8, 2009); 12/510,930 (filed Jul. 28, 2009); and 12/765,790 (filed Apr. 22, 2010). To the extent the foregoing applications and/or any other materials incorporated herein by reference conflict with the disclosure presented herein, the disclosure herein controls.

From the foregoing, it will be appreciated that specific embodiments of the technology have been described herein for purposes of illustration, but that various modifications may be made without deviating from the technology. For example, in other embodiments, other algorithms may be used to identify contacts that meet or exceed the performance criteria, while still employing a constrained number of contact combinations. The performance evaluation can be conducted on the basis of a single criterion or multiple criteria. Particular embodiments of the technology were described in the context of therapy applied to the lower thoracic vertebrae. In other embodiments, the therapy can be applied to other vertebrae. In still further embodiments, the therapy can be applied to neural populations other than those of the spinal cord. The foregoing technique can be used to select contacts on a lead having more or fewer than eight contacts (e.g., up to sixteen or more contacts, and/or down to four contacts). In some cases, the foregoing techniques can be used for multiple leads implanted near each other in the same patient, each of which can contain any number of contacts. If the contacts of one lead overlap those of another, the duplicate contacts or contact pairs can be exempted from testing during the trial period. While several techniques were described above in the context of an external programmer that communicates with a lead extending percutaneously from the patient to the programmer, other embodiments include other arrangements. Such arrangements include a programmer or other controller that is implanted within the patient and is attached to a fully implanted lead, and an external programmer or other controller that communicates wirelessly with a fully implanted lead.

Certain aspects of the technology described in the context of particular embodiments may be combined or eliminated in other embodiments. For example, the process of delivering modulation signals to at least one additional contact pair beyond n−1 can be eliminated in certain embodiments. Further, while advantages associated with certain embodiments have been described in the context of those embodiments, other embodiments may also exhibit such advantages and not all embodiments need necessarily exhibit such advantages to fall within the scope of the present technology. Accordingly, the present disclosure and associated technology can encompass other embodiments not expressly described or shown herein.

I/We claim:

1. A method for treating a patient, comprising:
implanting a signal delivery device proximate to a patient's spinal cord, the signal delivery device having eight contacts positioned to deliver neural modulation signals to the patient;
during a trial period:
(a) selecting a first contact;
(b) selecting a second contact immediately neighboring the first contact;
(c) delivering neural modulation signals to the patient via the first and second contacts;
(c) evaluating a performance characteristic of the neural modulation signals;
(d) repeating operations (a)-(c) with (i) a different first contact, (ii) a different second contact, or (iii) both a different first contact and a different second contact, until neural modulation signals have been applied to all pairs of neighboring contacts;
(e) without delivering neural modulation signals via any other combinations of contacts, selecting a pair of contacts for extended therapy; and
applying the neural modulation signals to the patient's spinal cord via the selected pair of contacts at a frequency of from about 3 kHz to about 50 kHz.

2. The method of claim 1 wherein selecting the first contact includes selecting a distal-most contact of the signal delivery device.

3. The method of claim 1 wherein the neighboring contacts are spaced apart from each other by at least one vertebral body.

4. The method of claim 1 wherein selecting the first contact includes selecting the first contact based at least on part on a vertebral level of the first contact.

5. The method of claim 1 wherein evaluating a performance characteristic includes evaluating an effectiveness with which the neural modulation signals reduce chronic pain in the patient.

6. The method of claim 1 wherein repeating operations (a)-(c) includes repeating operations (a)-(c) with (i) a different first contact, (ii) a different second contact or (iii) both a different first contact and a different second contact, selected in descending order of an expected value of the associated performance characteristic.

7. A method for treating a patient, comprising:
implanting a signal delivery device proximate to a patient's spinal cord, the signal delivery device having (n) contacts positioned to deliver neural modulation signals to the patient, where (n) is greater than three;
during a trial period:
(a) for each of at most (n−1) unique contact pairs, delivering first neural modulation signals to the patient and evaluating a performance characteristic of the neural modulation signals; and
(b) selecting a pair of contacts for further neural modulation, based at least in part on evaluating the performance characteristic of the first neural modulation signals; and
applying the first neural modulation signals or second neural modulation signals to the patient's spinal cord via the selected pair of contacts for an additional period of time.

8. The method of claim 7 wherein each of the unique contact pairs includes a first contact and an immediately neighboring second contact.

9. The method of claim 7 wherein at least one of the unique contact pairs includes a first contact and a non-neighboring second contact.

10. The method of claim 7 wherein evaluating includes evaluating for a period of at least one day before delivering the neural modulation signals to a different contact pair.

11. The method of claim 7 wherein (n) is equal to eight.

12. The method of claim 7 wherein immediately neighboring contacts of the signal delivery device are spaced apart by about one vertebral body.

13. The method of claim 7 wherein applying the first or second neural modulation signals includes applying neural modulation signals to address chronic pain in the patient.

14. The method of claim 7 wherein selecting a pair of contacts includes selecting a pair of contacts from a predetermined set of (n−1) contact pairs.

15. The method of claim 14 wherein the patient is one of multiple patients on whom the operations of implanting, delivering, selecting and applying are performed, and wherein selecting includes selecting from the same predetermined set of (n−1) contact pairs for each of the multiple patients.

16. A method for treating a patient, comprising:
implanting a signal delivery device proximate to a patient's spinal cord, the signal delivery device having (n) contacts positioned to deliver neural modulation signals to the patient, where (n) is greater than three;
during a trial period:
(a) for each of at most (n−1) unique contact pairs, delivering neural modulation signals to the patient and evaluating a performance characteristic of the neural modulation signals;
(b) based at least in part on evaluating the performance characteristic of the neural modulation signals delivered via the at most (n−1) unique contact pairs, and before delivering neural modulation signals to any other contact pairs, determining that none of the at most (n−1) unique contact pairs provides neural modulation with the performance characteristic meeting a performance criterion;
(c) for each of at least one additional contact pair beyond (n−1), delivering neural modulation signals to the patient and evaluating the performance characteristic of the neural modulation signals; and
(d) selecting a pair of contacts to deliver the neural modulation signals for an extended period of time, based at least in part on evaluating the performance characteristic of the neural modulation signals delivered via the at least one additional contact pair; and
applying the neural modulation signals to the patient's spinal cord via the selected pair of contacts for an extended period of time.

17. The method of claim 16 wherein applying neural modulation signals for an extended period of time includes addressing the patient's chronic pain.

18. The method of claim 16 wherein the performance criterion includes an effectiveness with which the neural modulation signals reduce chronic pain.

19. The method of claim 16 wherein selecting a pair of contacts includes selecting a pair of contacts from a predetermined set of (n−1) contact pairs.

20. The method of claim 19 wherein the patient is one of multiple patients on whom the operations of implanting, delivering, selecting and applying are performed, and wherein selecting includes selecting from the same predetermined set of (n−1) contact pairs for each of the multiple patients, regardless of patient condition.

21. A system for treating a patient, comprising:
a patient implantable signal delivery device having (n) contacts positioned to deliver therapy signals to a patient, where (n) is greater than three; and
an external signal generator coupled to the signal delivery device and having a computer-readable medium containing instructions that, when executed, perform the following operations:
(a) identify a contact pair;
(b) deliver neural modulation signals to the contact pair;
(c) change one or both of the contacts of the contact pair; and
(d) repeat operations (b)-(c) for each of at most (n−1) unique contact pairs.

22. The system of claim 21, further comprising a percutaneous signal delivery cable connected between the signal delivery device and the signal generator.

23. The system of claim 21, further comprising an implanted pulse generator programmed with instructions to apply neural modulation signals to the patient's spinal cord via a target pair of contacts for an extended period of time to address chronic pain in the patient.

24. The system of claim 21 wherein identifying a contact pair includes selecting a contact pair from a predetermined set of unique contact pairs.

25. The system of claim 21 wherein identifying a contact pair includes receiving a user input corresponding to at least one contact of the pair.

26. The system of claim 21 wherein identifying a contact pair includes identifying immediately neighboring contacts.

27. The system of claim 26 wherein changing one or both contacts includes changing from one immediately neighboring contact to another immediately neighboring contact.

28. The system of claim 21 wherein the computer-readable medium contains further instructions that, when executed perform the following operations:
for each of the contact pairs, receive an indication of a performance characteristic of the corresponding neural modulation signals; and
select a pair of contacts for extended neural modulation, based at least in part on the performance characteristic of the neural modulation signals corresponding to each of the contact pairs.

29. A system for treating a patient, comprising:
a computer-readable medium containing instructions that, when executed, perform the following operations:
(a) select a contact pair belonging to a set of (n) contacts carried by a patient-implantable signal delivery device, where n is greater than three;
(b) deliver neural modulation signals to the contact pair;
(c) change the contact pair; and
(d) repeat operations (b)-(c) for each of at most (n−1) unique contact pairs.

30. The system of claim 29 wherein the computer-readable medium contains instructions that, when executed, perform the following additional operations:
receive an indication of a deficient contact; and
automatically substitute another contact for the deficient contact.

31. The system of claim 29 wherein changing the contact pair includes automatically changing the contact pair.

32. The system of claim 29 wherein the computer-readable medium contains instructions that, when executed, perform the following additional operations:
for each of the contact pairs, automatically receive an indication of a performance characteristic of the corresponding neural modulation signals; and
select a pair of contacts for extended patient therapy, based at least in part on the performance characteristic indications.

33. A system for treating a patient, comprising:
a computer-readable medium containing instructions that, when executed, perform the following operations:
(a) select a contact pair belonging to a set of (n) contacts carried by a patient-implantable signal delivery device, where n is greater than three;
(b) deliver neural modulation signals to the contact pair;
(c) change the contact pair;
(d) repeat operations (b)-(c) for each of at most (n−1) unique contact pairs;
(e) receive an indication corresponding to none of the at most (n−1) unique contact pairs providing neural modulation with the performance characteristic meeting a performance criterion; and
(f) for each of at least one additional contact pair beyond (n−1), deliver neural modulation signals to the patient.

34. The system of claim 33 wherein n is at least eight.

35. The system of claim 33 wherein the computer-readable medium contains instructions, that, when executed, deliver neural modulation signals to at least one of the at least one additional contact pair for an extended period of time.

36. The system of claim 33 wherein the computer-readable medium contains instructions, that, when executed, automatically select each of the at least one additional contact pairs beyond (n−1).
